(19) United States
(12) Patent Application Publication
(10) Pub. No.: US 2006/0205024 A1
(43) Pub. Date: Sep. 14, 2006

(54) Method to diagnose and evaluate progression of Alzheimer's disease

(75) Inventors: Joseph Rogers, Glendale, AZ (US); Rena Li, Scottsdale, AZ (US); Diego Mastroeni, Surprise, AZ (US); Andrew Grover, El Mirage, AZ (US); Marwan Sabbagh, Scottsdale, AZ (US)

Correspondence Address: JENNINGS, STROUSS & SALMON, P.L.C., 201 E. WASHINGTON ST., 11TH FLOOR, PHOENIX, AZ, 85004, US

(73) Assignee: Sun Health Research Institute

(21) Appl. No.: 11/371,667

(22) Filed: Mar. 8, 2006

Related U.S. Application Data
(60) Provisional application No. 60/660,232, filed on Mar. 8, 2005.

(51) Int. Cl. G01N 33/537; G01N 33/53; G01N 33/543; G01N 33/00

(52) U.S. Cl. 435/7.92; 436/86

(57) ABSTRACT

A method or technique of detecting, diagnosing, monitoring, and evaluating a neurodegenerative disease, such as Alzheimer's disease, in a human body fluid is disclosed. The invention provides for a method of detecting amyloid peptides associated with erythrocytes and amyloid peptide complement complexes, as a diagnostic test for the detection, monitoring, evaluation or diagnosis of Alzheimer's disease and amyloid-based neurodegenerative diseases. More specifically the invention is directed to a method for detecting presence and amount of amyloid peptides such as amyloid beta (“Aβ”) and Aβ complement complexes, and related complement complexes and compounds with a role in neurodegenerative diseases, in an erythrocyte fraction of a human bodily fluid.

### CLAIM TO DOMESTIC PRIORITY

This Application claims the benefit of priority of U.S. Application Ser. No. 60/660,232, filed Mar. 8, 2005.

### FIELD OF THE INVENTION

The present invention relates to a method of detecting amyloids and amyloid complement complexes and to a diagnostic test for the detection, monitoring, or diagnosis of amyloid-linked neurodegenerative diseases. More specifically, the invention is directed to a method for detecting amyloid peptides, such as the peptide amyloid beta (“Aβ”), and related complement complexes and erythrocytes and to a diagnostic assay for Alzheimer's disease.

### BACKGROUND OF THE INVENTION

Neurodegenerative diseases of the human brain are typically linked with a condition of amyloidosis, or the formation of amyloid plaques on the brain, which limits cognitive function. For example, the most common form, Alzheimer's disease (“AD”), is a debilitative brain disease characterized by loss of memory, decline in balance and basic motor skills, as well as drastic decline in reasoning skills, judgment, and sense of orientation. AD typically progresses over a period of several years, ultimately resulting in complete impairment of multiple cognitive functions and eventually death.

Amyloid plaque deposits and the associated increased neuronal depolarization induced by the presence of amyloid peptides are also associated with other degenerative neurological diseases, injuries, and conditions. These include multiple sclerosis (“MS”), Parkinson's disease, multiple myeloma, Creutzfeldt-Jakob disease, macroglobulinemia, Huntington's disease, familial amyloid polyneuropathy and cardiomyopathy, systemic senile amyloidosis, familial amyloid polynephropathy, Down Syndrome, cerebral hemorrhages, familial amyloidosis, Gerstrnann-Straussler-Scheinker syndrome, Muckle-Wells syndrome, medullary carcinoma of thyroid, isolated atrial amyloid, and hemodialysis-associated amyloidosis, among others.

The diagnosis of neurodegenerative diseases is difficult and often inaccurate. Studies have shown, for example, that anywhere from approximately 10-30% of patients diagnosed with AD in the later stages of life were found to have been misdiagnosed upon autopsy. Diseases such as AD are often missed or misdiagnosed, because the diagnoses are performed using non-specific testing such as by CT scan, MRI, or electroencephalograph (EEG) to detect changes in the patient's brain. Typical testing may also include psychological or motor function testing. However, these tests can not provide an accurate diagnosis in all patients. More recently, genetic testing and mitochondrial DNA testing have been developed, but these are expensive and have not yet been evaluated thoroughly for accuracy.

Amyloid plaque deposits in the human brain associated with neurodegenerative diseases are typically characterized by aggregates of the peptide amyloid beta (“Aβ”) and other amyloid peptides. Aβ and similar amyloid peptides are considered to be causative precursors to the development of neurodegenerative diseases such as AD. These amyloid peptides are derived from the amyloid precursor protein, processed by proteases found in the human patient. In addition to deposits in the brain and cerebral blood vessels Aβ and similar amyloid peptides are also detectible in the peripheral circulatory system, tissues, excretions, and other human body fluids.

Aβ and other amyloid peptides are subject to a classic pathway for clearance of pathogens from the blood called immune adherence. It is well known that Aβ and other amyloid peptides can activate the complement system, or system of serum proteins circulating in blood plasma which interact in a sequential cascade. The basic complement pathway consists of a number of major protein components that become activated to destroy bound antigen-antibody complexes. In humans without neurodegenerative disease, cells are protected, at least in part, from complement activation by convertase inhibitors. For example, C3 convertase inhibitors are proteins that inhibit the cleavage of C3, a major protein component in the system. Through immune adherence, circulating pathogens become opsonized by complement molecules, and the pathogen/opsonin complexes become bound to erythrocytes, or red blood cells, where they are ferried to liver and spleen for degradation.

However, in humans with neurodegenerative disease, the complement system is activated, rendering the body unable to effectively deliver pathogens to the liver. For example, the protein C3 is cleaved into products including C3b and 1C3b which in turn activate the complement cascade. More specifically, the activation of several serine proteases leads to binding or adherence of complement opsonins, including C3b, to Aβ. For example, C3b may covalently attach to a pathogen surface and act as an opsonin in a “complement complex.” This “complement complex,” formed by the Aβ and the C3b, is capable of adhering to pathogens in blood and can “clear” or remove pathogens in the patient's system by binding to complement receptors on erythrocytes. In these patients, particularly individuals with AD, instead of moving toxins to the liver, these complement activation products become localized in lesions of the brain, and in other body tissues and fluids.

It is well known that Aβ and other amyloid peptides are detectible in erythrocyte fractions of blood. However, although immune adherence has been well-studied in the context of other pathogens for many years, it has not heretofore been suspected to play a role in AD or in Aβ clearance, nor has it been suspected that complement-opsonized Aβ or Aβ associated with erythrocytes might provide a reliable diagnostic and/or biomarker for AD. Instead, the presence of Aβ and other amyloid peptides in the erythrocyte compartment has been thought to reflect direct insertion of the amyloid peptides into the erythrocyte membrane. In fact, the association of Aβ and other amyloid peptides with plasma proteins and erythrocytes has been considered to be an impediment, rather than a critical key, to accurate quantification or immunoassay quantification of the amyloid peptides in blood. For example, as a result, rather than focus on the association of erythrocytes and plasma proteins with Aβ, prior studies have eliminated the association where Aβ in AD patients was measured.

Additionally, Aβ has been shown to bind to erythrocytes in human body fluids without the presence of an antibody. Aβ has also been shown to bind to other complement cascade products, such as C1q, and are detectible in human body fluids. Other amyloid peptides associated with one or more of the proteins in the complement system (e.g., forming an amyloid-complement complex) and other amyloid peptides associated with erythrocytes are also detectible in human body fluids. However, Aβ and Aβ-complement complexes, and other amyloid peptide-erythrocyte associations have never been considered in the context of a diagnostic or biomarker for AD in human body fluids.

Currently detection of neurodegenerative diseases such as AD requires extensive and expensive testing. Furthermore, the test results are often inconclusive, inaccurate, or involve highly invasive procedures. Therefore, there is a need for a rapid, cost-effective, non-invasive method of diagnosing neurodegenerative diseases such as AD, based upon a method of assaying human body fluids such as blood samples.

### DESCRIPTION OF THE FIGURES

FIG. 1A is a series of immunoassay photos illustrating co-localization of Aβ42 and an opsonin C3b on erythrocytes, 15 minutes (min) after spiking blood samples with 500 picogram per milliliter (pg/ml) Aβ42.

FIG. 1B is a series of immunoassay photos illustrating co-localization of Aβ42 and receptor CR1 on erythrocytes, 15 min after spiking blood samples with 500 pg/ml Aβ42.

FIG. 2A is an illustration of Mean±SEM C3-bound (i.e., C3 immunoprecipitated) Aβ42 in the erythrocyte compartment of 12 living Alzheimer's disease (AD) diagnosed subjects, 12 living mild cognitive impairment (MCI) subjects, and 12 living neurologically-normal elderly (ND) subjects, where Aβ42 concentrations in the erythrocyte pool were normalized to milliliter (ml) erythrocytes per ml blood.

FIG. 2B is an illustration of individual C3-bound (i.e., C3 immunoprecipitated) Aβ42 in the erythrocyte compartment of 12 living Alzheimer's disease (AD) diagnosed subjects, 12 living mild cognitive impairment (MCI) subjects, and 12 living neurologically-normal elderly (ND) subjects, where Aβ42 concentrations in the erythrocyte pool were normalized to ml erythrocytes per ml blood.

FIG. 3 is a graph illustrating the correlation of C3-bound (i.e., C3 immunoprecipitated) Aβ42 levels in the erythrocyte compartment to MMSE scores.

### DETAILED DESCRIPTION OF THE INVENTION

Amyloid peptides, such as beta amyloid peptide (Aβ), associated with degenerative neurological disorders, are found not only in the brain, but also in human body fluids such as blood. The present invention provides a method of detecting amyloid peptides associated with erythrocytes, and amyloid peptide-complement complexes, as a diagnostic in detecting, evaluating, monitoring and diagnosing degenerative neurological disorders such as Alzheimer's disease.

The present disclosure includes a method of detecting, monitoring and evaluating the progression of neurodegenerative disease, condition, or disorder, such as AD, in a human body fluid or tissue sample. In one aspect, the method includes performing an assay to detect the levels or presence of amyloid peptides associated with erythrocytes and/or complement molecules in a human body fluid or tissue, wherein the amyloid peptides are associated with a neurodegenerative disease, condition, or disorder. In another aspect, the method comprises evaluating or monitoring a patient for a neurodegenerative disease, condition, or disorder, based on the detection of levels or presence of amyloid peptides associated with erythrocytes and/or complement molecules in a human body fluid or tissue.

In one embodiment, the present disclosure provides for a method for detecting the level or presence of amyloid peptide associated with a complement complex, including amyloid peptides such as beta amyloid, serum amyloid p, amyloid a, and/or other amyloid peptides capable of associating or binding with a complement complex, as a diagnostic test or biomarker for a degenerative neurological disorder, or to monitor or evaluate disease progression. In an additional embodiment, the present disclosure provides for a method for detecting the level or presence of amyloid peptide associated with an erythrocyte, including amyloid peptides such as beta amyloid, serum amyloid p, amyloid a, and other amyloid peptides capable of associating or binding with an erythrocyte, as a diagnostic test or biomarker for a degenerative neurological disorder, or to monitor or evaluate disease progression.

Combinations of these two embodiments are also envisioned. For example, in an alternate embodiment, the present disclosure a method for detecting the level or presence of beta amyloid peptide-erythrocyte and beta amyloid peptide-C3b complement complexes present in an erythrocyte fraction of human blood as a diagnosis, evaluation, or monitoring of degenerative neurological disease progression, such as AD.

Finally, the present disclosure provides for a kit comprised an assay for detecting the level or presence of an amyloid peptide complement complex. The kit may also include a separate device for quantifying the amount of amyloid peptide complement complex present in a sample. However, the assay itself may be sufficient to quantify the amount of complex. The kit also includes instructions for diagnosing, evaluating, monitoring or testing for amyloidosis and/or any of the neurological conditions attributable to increase amyloidosis based on the results of the assay.

As used herein, the term “amyloid peptide” is understood to include a beta amyloid peptide (Aβ), a Aβ peptide-complement complex, serum amyloid p peptide, serum amyloid p peptide associated with an erythrocyte, serum amyloid p peptide associated with a complement complex, amyloid a peptide, serum amyloid a peptide associated with a complement complex, amyloid a peptide associated with an erythrocyte, an Aβ peptide associated with erythrocytes, an amyloid peptide capable of binding with an erythrocytes and associated with the erythrocyte, an amyloid peptide derived from an amyloid precursor protein by cleavage that is associated with amyloid plaque lesions in the human brain associated with an erythrocyte, an amyloid peptide capable of binding with a complement complex and associated with that complement complex, and an amyloid associated with erythrocytes, wherein the amyloid is derived from an amyloid precursor protein by cleavage and is associated with amyloid plaque lesions in the human brain.

As further used herein, the term “Aβ complement complex” or “amyloid peptide complement complex” includes Aβ-opsonin complexes, amyloid peptide-opsonin complexes, amyloid peptide-1C3b complexes, amyloid peptide-C3b complexes, Aβ-1C3b complexes, Aβ-C3b complexes, Aβ-C1q complexes, Aβ-C3b-factor B complexes, Aβ-convertase complexes, and amyloid peptide-complement complexes bound with additional molecules or complexes such as erythrocytes. As also used herein, the phrase “human body fluid” includes: human tissues that contain erythrocytes, cerebrospinal fluid, cellular material, organ tissues, saliva, blood, plasma, semen, vaginal fluid, mucous, urine, serum, bile, and the like.

In various embodiments, the terms “detecting an amyloid peptide” is understood to be performing an assay for one or more of the following detections: detecting amyloid peptide levels in a human body fluid, detecting the presence or absence of amyloid peptides in a human body fluid, detecting the amount of amyloid peptide adherence to complement complexes, such as C3b, in a human body fluid, detecting the amount of amyloid peptide adherence to an erythrocyte in a human body fluid, detecting the presence or absence of an amyloid peptide adherence to complement complexes, such as C3b, in a human body fluid, detecting the presence or absence of amyloid peptide adherence to a erythrocyte in a human body fluid, detecting the presence or absence of an amyloid peptide and a complement complex, such as C3b, in a human body fluid, and/or detecting the levels of an amyloid peptide and a complement complex, such as C3b, in a human body fluid.

As used herein, the term “detecting” is understood to mean deriving a value of an assay based on a radioactive, molecular weight, sequence, wavelength, chromatographic, fluorescent, numeric, mass, volume, visual, photographic, or other positive, negative, or scaled quantification of an assay result. Accordingly, in multiple embodiments of the invention, the derived value of the assay for detection results in an amyloid peptide assay measurement.

As used herein, “amyloid peptide assay measurement” includes measuring a positive result, measuring a negative result, or measuring detectable levels of amyloid peptide to derive a quantifiable value based on a radioactive, molecular weight, sequence, wavelength, chromatographic, fluorescent, numeric, mass, volume, visual, photographic, or other positive, negative, or scaled quantification of an assay result. “Amyloid peptide assay measurement” is further construed to include the presence of amyloid peptide bound with erythrocytes, the presence of amyloid peptide complement complex, the absence of amyloid peptide, the absence of the presence of amyloid peptide complement complex, the level of amyloid peptide complement complex, the level of amyloid peptide, the amount of amyloid peptide, and/or the amount of amyloid peptide complement complex.

“Comparison” or “evaluation” of an “amyloid peptide assay measurement” as used herein is further understood to include comparing or evaluating an amyloid peptide measurement with a range of measurements or a standard scale or table for neurodegenerative diseases. “Evaluation” or “comparison” as it is used herein also includes analyzing data obtained from a human individual in relation to a group of other individuals, as a value, range, scale, or table, based on neurocognitive stage or diagnosis.

For example, the value, range, scale or table, may have numeric values, or may include multiple categories such as “positive” or “negative,” “normal,” “mild cognitive impairment,” “AD” or other disease, which correspond to values or stages of disease progression or diagnosis. These values, ranges, scales or tables correspond to a positive or negative diagnosis of a neurological condition, disease, or disorder associated with increased neuronal depolarization induced by the presence of an amyloid peptide.

As is also used herein, the term “monitoring” is understood to include a course of observation, evaluation, diagnosis, treatment or on-going review of neurological state or disease progression, or systematic testing of an individual, group, or population for a neurodegenerative disease. For example, monitoring would include a continued course of testing for an individual diagnosed with mild cognitive impairment for progression to Alzheimer's disease, over a period of time.

In the most basic embodiment, the detection aspect of the method comprises detecting an amyloid peptide associated in a human body fluid sample, wherein detection is an indicator for a specific degenerative neurological condition. Within various embodiments, detecting an amyloid peptide may include detection of an amyloid peptide associated with an erythrocyte, detection of an amyloid peptide associated with a complement complex, or detecting an amyloid peptide associated with both a complement complex and an erythrocyte. In various basic embodiments, the method comprises detecting the amyloid peptide in the erythrocyte compartment of human blood, or detecting the amyloid peptide-complement complex in the erythrocyte compartment of human blood or other body fluid, for example, such as detecting the presence of amyloid peptide-erythrocyte associations in other human body fluids such as plasma, spinal fluid, serum, or urine.

In a more specific embodiment, the detection aspect of the method comprises detecting a beta amyloid peptide (Aβ) and a beta amyloid peptide Aβ-C3b complement complex in a human body fluid sample, wherein detection is an indicator for a specific degenerative neurological condition such as AD. In another specific embodiment, the detection aspect of the method comprises detecting a beta amyloid peptide Aβ-C3b complement complex in a human body fluid sample, wherein detection is an indicator for a specific degenerative neurological condition such as AD. In further embodiment, the method comprises detecting Aβ in the erythrocyte compartment of blood in combination with detecting Aβ in other blood plasma or serum components.

In one example, Aβ complexes with erythrocyte-bound complement at a receptor (e.g., receptor CR1) are measured using an assay, in order to detect the levels of Aβ-C3b “complement complex” in the human body fluid. In another example, Aβ-C3b “complement complexes” associated with erythrocytes are measured to detect the presence or absence of the “complement complexes.” In this embodiment the method of evaluation comprises analyzing the measurements levels of Aβ or Aβ-C3b complement complexes. These values that are obtained are then used as a diagnostic for AD or a biomarker for neurological disease progression.

In various embodiments, the assay aspect of the present method comprises any technique or combination of techniques capable of detecting or measuring the presence, absence, concentration, or amount of an amyloid peptide, amyloid peptide complement complex, or amyloid peptide/erythrocyte association.

In one embodiment, the assay detects the presence of amyloid peptide in a bodily fluid wherein the amyloid peptide is bound by complement opsonin, such as C3b, and/or is associated with erythrocytes (e.g., assaying blood and cerebrospinal fluid to detect the presence of Aβ complement complexes using a combination of immunoprecipitation and ELISA assays). For example, assays capable of detecting amyloid peptides include ELISA assay using anti-complement antibodies for capture and anti-Aβ antibodies for detection (or vice-versa), Western blotting, mass spectroscopy, flow cytometry, measurement Aβ associated with erythrocytes, or by other methods.

In another embodiment the assay includes characterization of amyloid peptides in a body fluid. These techniques include, but are not limited to, structural analysis such as spectrophotometry, peptide solubilization and aggregation, HPLC purification, such as a fractionation by reverse phase HPLC, or Western Blot Analysis, such as to measure the level of oligomerization of different amyloid peptides and assess immunoreactivity, e.g., using enhanced chemiluminescence (ECL).

In a further embodiment, the assay measures complement activation, to detect complement-adherent amyloid peptides in a bodily fluid such as blood, or to detect amyloid peptides associated with erythrocytes as a diagnostic detection or biomarker for a degenerative neurological disease, such as AD, for example, detection by hemolytic assay to measure the activity of the basic complement pathway, or quantification of activation products by ELISA assay. In yet another embodiment of the invention, the method includes an assay to measure the binding of amyloid peptides to complement complexes, such as C3b, such as by quantifying fluorescence of ELISA assay results by photometer. In yet another embodiment of the invention, the method includes an assay to measure the binding of amyloid peptides associated with erythrocytes, such as by amino acid sequencing.

In various additional aspects, the method further comprises evaluating the amyloid peptide assay measurement, wherein the evaluation comprises comparing the amyloid peptide assay measurement to amyloid peptide assay measurements for a human without the condition associated with increased neuronal depolarization induced by the presence of amyloid peptide.

For example, in one specific embodiment, the method comprises correlating detected levels of Aβ in the erythrocyte compartment of a human donor's blood, particularly Aβ complexes with complement that are bound to erythrocytes through complement receptor CR1, and deriving a numeric value for the donor via assay. In this embodiment, the numeric value for the donor is then compared to a known range of numeric values that correspond with cognitive ability. In this embodiment, the assay measures Aβ that has formed complexes with complement opsonins, such as C3b, in bodily fluids, or Aβ or Aβ/complement complexes associated with erythrocytes to derive a value. These values may be used in diagnosis or monitoring of a patient. For example, values are significantly lower in AD patients, somewhat higher with cognitive status measures of mild cognitive impairment (MCI), and higher still in normal elderly control subjects. The levels of Aβ or Aβ/complement complexes that are obtained are therein used as a diagnostic for AD or as a biomarker for the disease's progression in an individual. In various embodiments, the method further includes diagnosing, monitoring, or treating a degenerative neurological condition such as AD.

The following examples further demonstrate the methods of the present invention based on the general concepts that amyloid peptides and amyloid peptide complement complexes present in human body fluids may be measured by an assay to provide a statistically reliable diagnostic for AD. Furthermore, the following examples show that levels of the amyloid peptide and amyloid peptide complement complexes correlate with cognitive status, as a biomarker of AD disease progression, which is useful in evaluating and diagnosing an individual with a neurodegenerative disease, condition, or disorder.

### Example 1

### Detection of Aβ in a Human Blood Sample Using Immunocytochemical Assays

Experiments were performed to test C3b adherence of a commercially available beta amyloid peptide, Aβ-42 (Bachem, Bubendorf, Switzerland), to a receptor, CR1, on erythrocytes using blood samples taken from three normal healthy adult donors without known neurodegenerative disease. Blood samples were spiked in vitro with exogenous Aβ42, a beta amyloid peptide known to be strongly associated with Alzheimer's disease. Anticoagulants were added to derive clean erythrocyte samples. However, anticoagulants, such as EDTA, block complement activation and opsonization, so in order to prevent coagulation, two 5 ml tubes of blood were taken from each donor. This procedure permitted activation of complement in the serum by the newly introduced Aβ, with adherence of complement opsonins to the Aβ in the sample tube. As a control, parallel serum samples from the same blood sample in a second tube were also spiked with EDTA, which does not permit complement activation and adherence to occur.

The first tube was treated with EDTA and processed to separate out the erythrocyte fraction from the remainder of the plasma fraction. The erythrocyte fraction was retained and the plasma fraction discarded. The second tube was untreated and did not contain EDTA. Blood in the first tube was allowed to coagulate and then spun down to separate out the serum from erythrocytes and other material. The serum fraction was retained and the erythrocytes and other material were discarded. Serum from the second tube was spiked with 500 pg/ml to 500 ng/ml Aβ42 in the presence (EDTA-treated condition) or absence (EDTA-free condition) of 2 mM EDTA, a saturating concentration that essentially abolishes complement activation and provides a control for nonspecific binding of Aβ42 to erythrocytes.

After incubation at 37° C. for 15 minutes, the serum samples were recombined with equal volumes of erythrocytes from the first tube to permit binding of complement-adherent Aβ1-42 to erythrocytes. By this means, it was possible to obtain erythrocytes that were free of coagulated material while permitting complement activation and opsonization to take place in the absence of anticoagulant complement inhibitors (EDTA-free condition). The pooled serum/erythrocyte samples were then used in subsequent immunocytochemical assays.

To assess the immunochemistry, serum/erythrocyte samples were centrifuged at 800 g for 10 min at 4° C., serum was removed, and the erythrocyte fraction was re-suspended in 4% buffered paraformaldehyde for 10 min at 4° C. Fixative was removed by spinning at 800 g for 10 min at 4° C. The erythrocytes were then fixed to standard glass slides using a 1:1 acetone:ethanol mixture, which was allowed to evaporate overnight.

Immunoreaction was performed by a 1:200 dilution of mouse monoclonal anti-C3b antibody (Abcam, Cambridge, Mass.), a 1:1000 dilution of mouse monoclonal anti-CR1 antibody (Biomeda, Foster City, Calif.), or a 1:1000 dilution of rabbit polyclonal anti Aβ42 antiserum (Chemicon, Temecula, Calif.). After incubation for 3 hours at 4° C., anti-rabbit secondary antibody conjugated with Alexa Fluor 488 (Molecular Probes, Carlsbad, Calif.) was applied for 1 hour to label Aβ42 immunoreactivity, followed by a second incubation with anti-mouse secondary antibody conjugated with Texas Red (Vector, Burlingame, Calif.) for 3 hours at 4° C. to detect CR1 or C3b immunoreactivity. Slides were visualized using an Olympus confocal microscope.

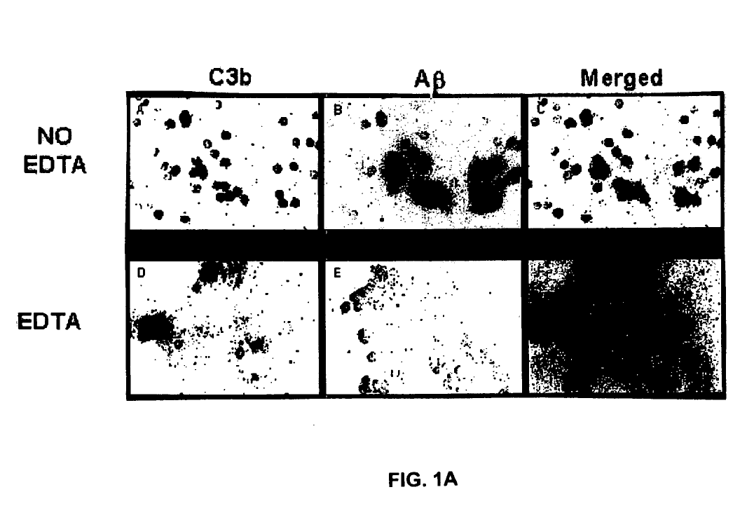

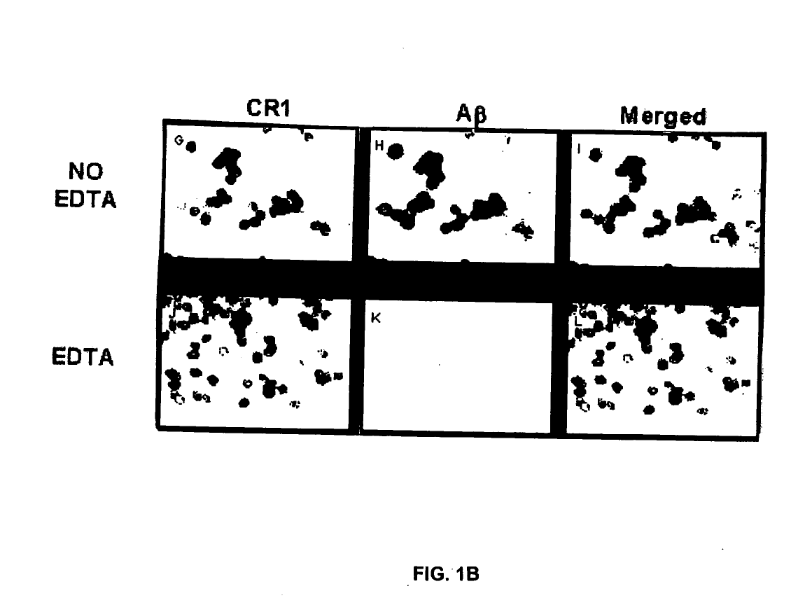

As shown in FIGS. 1A and 1B, the visualized fluorescent markers corresponded to a positive result for a component. As shown in FIG. 1A, the left column photos illustrate C3b complement opsonin results for both EDTA and non-EDTA trials, which were visualized by a red fluorophore (photos A and D). Also in FIG. 1A, the middle column photos indicate positive detection for Aβ 1-42, visualized by green fluorophors (photos B and E) for both EDTA and non-EDTA trials. FIG. 1A further illustrates the Aβ1-42-C3b complement complex, in the right column (photos C and F) visualized by mixed color fluorophors for both EDTA and non-EDTA trials. As shown in FIG. 1B, the left column photos illustrate CR1 receptor results for both EDTA and non-EDTA trials, which were visualized by a red fluorophore (photos G and J). Also in FIG. 1B, the middle column photos indicate positive detection for Aβ1-42, visualized by green fluorophors (photos H and K) for both EDTA and non-EDTA trials. FIG. 1B further illustrates Aβ1-42-CR1 complexes, in the right column (photos I and L) visualized by mixed color fluorophors for both EDTA and non-EDTA trials.

As also shown in FIG. 1A, Aβ in the blood rapidly associated with complement opsonins such as C3b. Additionally, FIG. 1A shows that these complement opsonin/Aβ complexes were then rapidly bound to erythrocytes, such that the complement opsonin C3b. As shown in FIG. 1B, Aβ, and the complement receptor CR1 were be co-localized together at the surface of erythrocytes. In non-EDTA samples where complement activation and adherence to Aβ could occur, Aβ immunoreactivity was readily detected at the surface of erythrocytes (FIG. 1A, photo B), and this immunoreactivity co-localized with immunoreactivity for complement C3b (FIG. 1A, photos A and C). By contrast, in EDTA-treated samples where complement activation and adherence to Aβ could not occur, only faint Aβ immunoreactivity at the erythrocyte surface could be detected, and then only after digital enhancement (FIG. 1A, photo E). Moreover, the marginal Aβ immunoreactivity did not co-localize with complement C3b (FIG. 1A, photos D and F). Similarly, under non-EDTA conditions, Aβ could be co-localized with erythrocyte CR1 at the erythrocyte surface (FIG. 1B, photos G and I). However, this did not occur under conditions when EDTA was present (FIG. 1B, photos J and L).

### Example 2

### Detection of Aβ in Human Blood Samples Using ELISA Assays

Studies were also performed in order to measure beta amyloid peptide Aβ42, where the peptides had already been bound by complement prior to collection. Blood samples were taken from elderly patients, in several groups, based upon previous known diagnosis of degenerative neurological condition. Twelve subjects carried the antemortem diagnosis of possible or probable AD (AD). Twelve subjects carried the antemortem diagnosis of mild cognitive impairment (MCI). Twelve subjects carried the antemortem diagnosis of being non-demented, neurologically-normal elderly (ND). After Institutional Review Board approval of the protocol, 36 human subjects were recruited and 7-9 ml of blood was drawn from each by standard venipuncture. All subjects received thorough antemortem evaluation using the protocols of the National Institute on Aging Alzheimer's Disease Centers. Blood samples from three additional normal healthy adult donors not known to have degenerative neurological disorders were also taken by venipuncture for in vitro experiments.

Patient blood samples were collected in EDTA-treated tubes to prevent coagulation, and numbered under blind study conditions. After centrifugation at 800 g for 10 min at 4° C., the plasma and buffy coat were removed and stored at −80° C. until all samples had been collected. To lyse the remaining erythrocyte fraction, each was suspended in five volumes of ice-cold distilled water that included one tablet Complete Protease Inhibitor (Roche, Indianapolis, Ind.) per 50 ml distilled water, inverted ten times, and incubated packed in ice for 30 minutes. The lysed erythrocytes were then spun at 3,300 g for 10 minutes at 4° C. The erythrocyte membrane pellet was re-suspended in 1.2 ml of 0.1% Triton X100 (in 50 mM Tris buffer, pH 8.0), vortexed on low for 10 seconds, and centrifuged at 3,300 g for 10 minutes at room temperature. The supernatant was removed and stored at −80° C. Volumes of each blood sample and the volumes of the plasma and erythrocyte fractions were then recorded.

When all patient samples had been accumulated, the processed erythrocyte samples were thawed and aliquoted into 500 μl duplicates, to each of which 5 μl of goat anti-C3 antiserum (Advanced Research Technology, San Diego, Calif.) was added. The duplicates were then individually loaded into disposable immunoprecipitation columns (Protein G IP Kit) (Sigma, St. Louis, Mo.) and incubated overnight at 4° C. with head-tail inversion. To each column, 30 μl of protein G agarose (Sigma, St. Louis, Mo.), pre-washed with 1 ml of IX immunoprecipitation buffer was added according to the manufacturer protocol for Protein G IP Kit, (Sigma, St. Louis, Mo.). This was followed by a second overnight incubation at 4° C. with head-tail inversion.

After the erythrocyte and plasma fractions were separated, the erythrocytes were lysed, and the erythrocyte membranes were solubilized to release any ligands bound to them. The supernatants containing such ligands were then subjected to immunoprecipitation with an antiserum directed at C3 to capture C3-related opsonins such as C3b and any molecules bound by them. More specifically, after centrifugation at 12,000 g for 30 seconds at 4° C., the effluents were saved to measure Aβ that was not bound to C3 opsonins. The columns were then washed six times with 700 μl of immunoprecipitation buffer and one time with 700 μl of 0.1× immunoprecipitation buffer. To elute C3-opsonized proteins from the columns, 200 μl of a 0.1 M glycine solution (adjusted to pH3.5 with HCl) was added, followed by centrifugation at 12000 g for 30 seconds at 4° C. In order to neutralize pH of the eluents for subsequent ELISA assay, 20 μl of 1 M Tris buffer (pH 8.5) was added to each 200 μl aliquot of the immunoprecipitated erythrocyte material.

Plasma and erythrocyte samples were then subjected to an Aβ42 specific ELISA assay using commercial 96-well Aβ42 ELISA kits with Super-Sensitive Secondary Detection (Amersham Biosciences, Piscataway, N.J.) according to manufacturer's protocol. Wells were loaded with 100 μl of sample each. To account for slight differences in erythrocyte volumes and the amounts of blood collected per subject, Aβ42 values were expressed as pg/ml erythrocytes/ml blood when comparisons of the experimental groups were performed. Aβ42 concentrations in the erythrocyte pool were normalized to ml of erythrocytes per ml of blood where the percentage of blood occupied by the erythrocyte fraction varied from patient to patient. Simple expression of Aβ42 values as pg/ml erythrocytes did not statistically or materially affect the results.

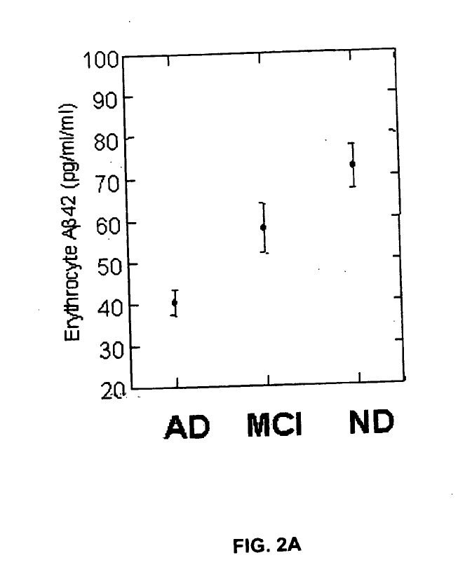

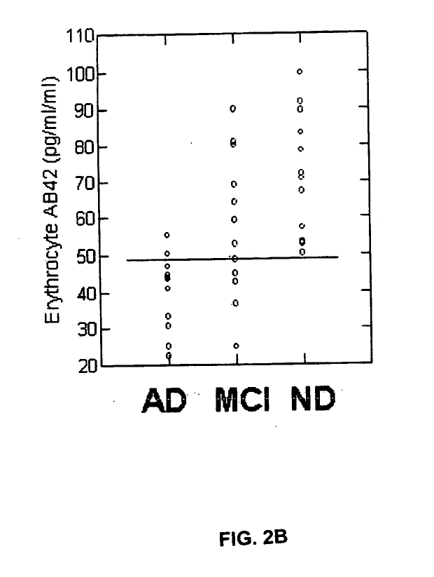

In analyzing the results, any Aβ 1-42 detected in the ELISA was assumed to be bound to complement, because otherwise it could not have been captured by the complement immunoprecipitation. Moreover, the Aβ 1-42 was further assumed to have been associated with the erythrocyte fraction, as this was the only fraction of blood assayed. As shown in FIGS. 2A and 2B, complement adherence to Aβ and Aβ binding to erythrocytes occurred and was detectible with an ELISA assay as a diagnostic for detection of neurological diseases such as AD.

As shown in FIG. 2A, Mean values for AD patients were significantly lower than those for MCI (t=2.76, P=0.014), and ND (t=5.67, P<0.0001) subjects. MCI patients also exhibited lower mean erythrocyte Aβ42 levels than ND subjects at values that approached significance (t=1.93, P=0.067), as also shown in FIG. 2A. FIG. 2B provides individual measures of the amounts of complement-adherent erythrocyte Aβ1-42 for each patient after normalization to the amounts of blood in each sample and the volumes of erythrocytes in each sample. Results indicated that not only did the AD group differ significantly from the normal elderly group on average, as shown in FIG. 2A, but also that there was little overlap of the data for AD and normal elderly controls on an individual basis, as shown in FIG. 2B.

These results are consistent with missed diagnosis or misdiagnosis of neurological disorders in elderly patients. Using the general statistic of 10-30% missed or misdiagnosis, the expected results for 12 individuals would place one to three such patients outside of the range for AD subjects and into the range for normal subjects. The results also indicated that two AD patients had Aβ levels in the normal range. The results also found two normal subjects had Aβ levels in the high range of AD. This was also consistent with progression and pattern of the disease. Approximately 50% of MCI patients, or those with mild cognitive impairment, which have not yet meet full criteria for the diagnosis of AD, convert to the diagnosis of AD within five years. Thus, with a perfectly accurate diagnostic, one would expect approximately 50% of MCI patients to fall within the range of AD patients, as is also demonstrated in the results.

### Example 3

### Method for Detecting Neurodegenerative Disease Progression

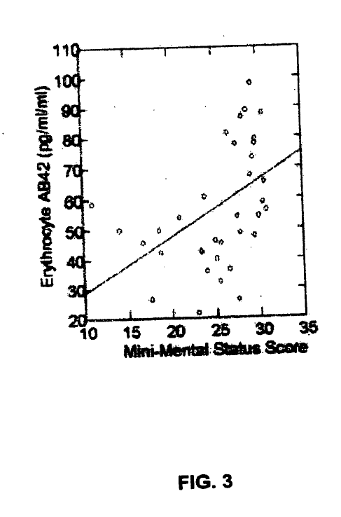

Following the protocol disclosed in Example 2, the method of the present invention was performed a biomarker for AD disease progression. Mini-Mental Status Evaluation (MMSE) scores, a common measure of cognitive decline in AD, were obtained for the patients and compared to their concentrations of complement-opsonized erythrocyte Aβ 1-42 after normalization to blood sample and erythrocyte volumes as shown in FIG. 3. A significant correlation was obtained (R=0.446, P=0.006), indicating that the measure has significant predictive value as a biomarker for deficits in cognitive status that occur in AD.

Overall, the results demonstrate that Aβ and Aβ-complement complexes in human body fluids are detectable using an assay, and that measurements taken from an assay provide a statistically reliable diagnostic for AD. Experimental data demonstrates that levels of the Aβ and Aβ-complement complexes correlate with cognitive status, consistent with application as a biomarker of AD disease progression.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of a preferred embodiment and best mode of the invention known to the applicant at this time of filing the application has been presented and is intended for the purposes of illustration and description. It is not intended to be exhaustive nor limit the invention to the precise form disclosed and many modifications and variations are possible in the light of the above teachings. The embodiment was chosen and described in order to best explain the principles of the invention and its practical application and to enable others skilled in the art to best utilize the invention in various embodiments and with various modifications as are suited to the particular use contemplated. Therefore, it is intended that the invention not be limited to the particular embodiments disclosed for carrying out the invention.

What is claimed:

1. A method of detecting the presence of amyloid plaques in the brain of an animal indicative of a degenerative neurological condition, comprising:
performing an assay on a sample of bodily fluid from an animal, wherein the assay is capable of detecting an amount of amyloid peptide complement complex in the sample; and
determining the amount of amyloid peptide complement complex in the sample;
comparing the amount of amyloid peptide complement complex to a predetermined range of amounts of amyloid peptide complement complexes in animals; and
arriving at a result based on the comparison, wherein the result is predictive of a level of amyloidosis, the level of amyloidosis being indicative of the presence or absence of a degenerative neurological condition.

2. The method of claim 1, wherein the assay is selected from the group consisting of: an ELISA, Western Blot Analysis, mass spectroscopy, flow cytometry, spectrophotometry, peptide solubilization and aggregation, HPLC purification, and fractionation by reverse phase HPLC.

3. The method of claim 1, wherein the bodily fluid is selected from a group consisting of: blood, serum, erythrocyte-containing tissue, erythrocyte fraction of any fluid, plasma, urine, and cerebrospinal fluid.

4. The method of claim 1, wherein the amount is determined by deriving a value from the assay based on a quantification selected from the group consisting of: radioactive, molecular weight, nucleotide sequencing, amino acid sequencing, wavelength, chromatographic, fluorescent, numeric, mass, volume, visual and photographic.

5. The method of claim 1, wherein the amyloid peptide complement complex is selected from the group consisting of: an amyloid peptide associated with a complement, a serum amyloid a peptide associated with a complement, a serum amyloid p peptide associated with a complement, an amyloid peptide associated with an erythrocyte, an amyloid peptide associated with an opsonin, an amyloid peptide complement complex associated with an erythrocyte, an Aβ-opsonin complex, an amyloid peptide-opsonin complex, an amyloid peptide-1C3b complex, an amyloid peptide-C3b complex, an Aβ-1C3b complex, an Aβ-C3b complex, an Aβ-C1q complex, an Aβ-C3b-factor B complex, an Aβ-convertase complex, and an amyloid peptide associated with a complement and any another molecule.

6. The method of claim 1, wherein the result is also predictive of the severity of the amyloidosis.

7. The method of claim 6, wherein the severity of the amyloidosis is indictative of the severity of the degenerative neurological condition.

8. The method of claim 1, wherein the degenerative neurological condition is selected from a group consisting of: Alzheimer's disease, multiple sclerosis, Parkinson's disease, multiple myeloma, Creutzfeldt-Jakob disease, macroglobulinemia, Huntington's disease, familial amyloid polyneuropathy, familial amyloid cardiomyopathy, systemic senile amyloidosis, familial amyloid polynephropathy, Down Syndrome, cerebral hemorrhages, familial amyloidosis, Gerstmann-Straussler-Scheinker syndrome, Muckle-Wells syndrome, medullary carcinoma of thyroid, isolated atrial amyloid, and hemodialysis-associated amyloidosis.

9. The method of claim 1, wherein the animal is a human.

10. The method of claim 1, further including comparing to the result to a previous result for the animal.

11. The method of claim 10, further including using the comparison of the result to the previous result to monitor any progression in the degenerative neurological condition.

12. A kit for detecting the presence of amyloid plaques in the brain of an animal indicative of a degenerative neurological condition, comprising:
an assay capable of detecting an amount of amyloid peptide complement complex in a sample of bodily fluid from an animal; and
instructions including predetermined ranges of amounts of amyloid peptide complement complexes in animals, wherein the predetermined ranges are capable of being compared with a result from the assay, the comparison being predictive of the presence or absence of a degenerative neurological condition.

13. The kit of claim 12, further including a device capable of quantifying the amount of amyloid peptide complement complex in the sample.

14. The kit of claim 12, wherein the assay is selected from the group consisting of: an ELISA, Western Blot Analysis, mass spectroscopy, flow cytometry, spectrophotometry, peptide solubilization and aggregation, HPLC purification, and fractionation by reverse phase HPLC.

15. The kit of claim 12, wherein the bodily fluid is selected from a group consisting of: blood, serum, erythrocyte-containing tissue, erythrocyte fraction of any fluid, plasma, urine, and cerebrospinal fluid.

16. The kit of claim 12, wherein the amount is determined by deriving a value from the assay based on a quantification selected from the group consisting of: radioactive, molecular weight, nucleotide sequencing, amino acid sequencing, wavelength, chromatographic, fluorescent, numeric, mass, volume, visual and photographic.

17. The kit of claim 12, wherein the amyloid peptide complement complex is selected from the group consisting of: an amyloid peptide associated with a complement, a serum amyloid a peptide associated with a complement, a serum amyloid p peptide associated with a complement, an amyloid peptide associated with an erythrocyte, an amyloid peptide associated with an opsonin, an amyloid peptide complement complex associated with an erythrocyte, an Aβ-opsonin complex, an amyloid peptide-opsonin complex, an amyloid peptide-1C3b complex, an amyloid peptide-C3b complex, an Aβ-1C3b complex, an Aβ-C3b complex, an Aβ-C1q complex, an Aβ-C3b-factor B complex, an Aβ-convertase complex, and an amyloid peptide associated with a complement and any another molecule.

18. The kit of claim 12, wherein the degenerative neurological condition is selected from a group consisting of: Alzheimer's disease, multiple sclerosis, Parkinson's disease, multiple myeloma, Creutzfeldt-Jakob disease, macroglobulinemia, Huntington's disease, familial amyloid polyneuropathy, familial amyloid cardiomyopathy, systemic senile amyloidosis, familial amyloid polynephropathy, Down Syndrome, cerebral hemorrhages, familial amyloidosis, Gerstmann-Straussler-Scheinker syndrome, Muckle-Wells syndrome, medullary carcinoma of thyroid, isolated atrial amyloid, and hemodialysis-associated amyloidosis.

19. The kit of claim 12, wherein the animal is a human.

20. A method for diagnosing Alzheimer's disease in a human, comprising
performing an assay on a sample of bodily fluid from a human, wherein the assay is capable of detecting an amount of amyloid peptide complement complex in the sample; and
determining the amount of amyloid peptide complement complex in the sample;
comparing the amount of amyloid peptide complement complex to a predetermined range of amounts of amyloid peptide complement complexes in humans; and
arriving at a result based on the comparison, wherein the result is predictive of the presence or absence of Alzheimer's disease in the human.

21. The method of claim 20, wherein the result is predictive of severity of the Alzheimer's disease in the human.

22. The method of claim 20, wherein the assay is selected from the group consisting of: an ELISA assay, Western Blot Analysis, mass spectroscopy, flow cytometry, spectrophotometry, peptide solubilization and aggregation, HPLC purification, and fractionation by reverse phase HPLC.

23. The method of claim 20, wherein the amyloid peptide complement complex is selected from the group consisting of: an amyloid peptide associated with a complement, a serum amyloid a peptide associated with a complement, a serum amyloid p peptide associated with a complement, an amyloid peptide associated with an erythrocyte, an amyloid peptide associated with an opsonin, an amyloid peptide complement complex associated with an erythrocyte, an Aβ-opsonin complex, an amyloid peptide-opsonin complex, an amyloid peptide-1C3b complex, an amyloid peptide-C3b complex, an Aβ-1C3b complex, an Aβ-C3b complex, an Aβ-C1q complex, an Aβ-C3b-factor B complex, an Aβ-convertase complex, and an amyloid peptide associated with a complement and any another molecule.
